(19) United States
(12) Patent Application Publication
(10) Pub. No.: US 2020/0186880 A1
(43) Pub. Date: Jun. 11, 2020

(54) ELECTRONIC INSTRUMENT, RECORDING MEDIUM, CONTROL DEVICE, AND CONTROL METHOD

(71) Applicant: SHARP KABUSHIKI KAISHA, Sakai City, Osaka (JP)

(72) Inventors: MEGUMI YOKOGAWA, Sakai City, Osaka (JP); TADAO ANDOH, Sakai City, Osaka (JP); KIYOFUMI OHTSUKA, Sakai City, Osaka (JP)

(21) Appl. No.: 16/617,485

(22) PCT Filed: Apr. 6, 2018
(86) PCT No.: PCT/JP2018/014796

§ 371 Date: Nov. 26, 2019

(30) Foreign Application Priority Data
May 31, 2017 (JP) 2017-108761
Aug. 10, 2017 (JP) 2017-155907

(51) Int. Cl. H04N 21/45; A61B 5/16; H04N 21/258

(52) U.S. Cl. CPC H04N 21/4532; H04N 21/25891; A61B 5/165

(57) ABSTRACT

The present invention presents appropriate information that corresponds to a situation of a game which a user is watching arid reacting to. In an electronic apparatus (10) including information presenting devices (12, 15, 16) and a control device (11), the control device (11) controls the information presenting devices (12, 15, 16) to present information in response to the situation of the game which situation has been determined based on information that (i) relates to the game which the user is watching and reacting to and (ii) is accumulated in a server (30).

### TECHNICAL FIELD

The present invention relates to an electronic apparatus, a program, a control device, and a control method.

### BACKGROUND ART

Conventionally, research and development have been carried out in regard to an electronic apparatus that (a) estimates a feeling of a user based on information such as a user's facial expression and then (b) outputs an image, an audio message, and/or the like so as to appropriately respond to the feeling thus estimated. For example, Patent Literature 1 discloses an electronic apparatus which includes (i) a user state analyzing section for analyzing a state of a user, a content information analyzing section for analyzing a state of content, (iii) a comment preparing section for preparing, based on the user state and content state thus analyzed, a comment that is supposed to drew sympathy of the user, and (iv) a comment outputting section for outputting the comment thus prepared, by audio or the like.

### CITATION LIST

### Patent Literature

[Patent Literature 1]

Japanese Patent Application. Publication Tokukai No. 2016-186741 (Publication date: Oct. 27, 2016)

### SUMMARY

### Technical Problem

According to the technique disclosed in Patent Literature 1, information is presented based on the state of user and the state of content. However, the state of user varies from individual to individual, and all users do not therefore sympathize with the information thus presented. Meanwhile, in a case where only content, for example, a situation of a game which the user is watching and reacting to is used, accurate information cannot be presented to the user.

An object of an aspect of the present invention is to present appropriate information that corresponds to a situation of a game which the user is watching and reacting to.

### Solution to Problem

In order to attain the object, an electronic apparatus in accordance with an aspect of the present invention includes: an information presenting device; and a control device, the control device controlling the information presenting device to present information in response to a situation of a game which situation has been determined based on information that (i) relates to the game which a user is watching and reacting to and (ii) is accumulated in a server.

In order to attain the object, a control device in accordance with an aspect of the present invention is a control device for controlling an electronic apparatus and controls an information presenting device included in the electronic apparatus to present information in response to a situation of a game which situation has been determined based on information that (i) relates to the game which a user is watching arid reacting to and is accumulated in a server.

In order to attain the object, a control method in accordance with an aspect of the present invention is a method for controlling an electronic apparatus and includes the step of controlling an information presenting device included in the electronic apparatus to present information in response to a situation of a game which situation has been determined based on information that (i) relates to the game which a user is watching and reacting to and (ii) is accumulated in a server.

### Advantageous Effects of Invention

According to an aspect of the present invention, it is possible to bring about an effect of presenting appropriate information that corresponds to a situation of a game which the user is watching and reacting to.

### BRIEF DESCRIPTION OF DRAWINGS

FIG. 1 is a block diagram illustrating a main part configuration of a control device that is included in a wearable terminal in accordance with Embodiment 1 of the present invention.

FIG. 2 is a block diagram illustrating configurations of the wearable terminal and a smart phone in accordance with Embodiment 1 of the present invention.

FIG. 3 is a schematic view illustrating an appearance of the wearable terminal in accordance with Embodiment 1 of the present invention.

FIG. 4 is a flowchart showing processes carried out by the control device included in the wearable terminal in accordance with Embodiment 1 of the present invention.

### DESCRIPTION OF EMBODIMENTS

### Embodiment 1

The following description will discuss in detail Embodiment 1 of the present invention.

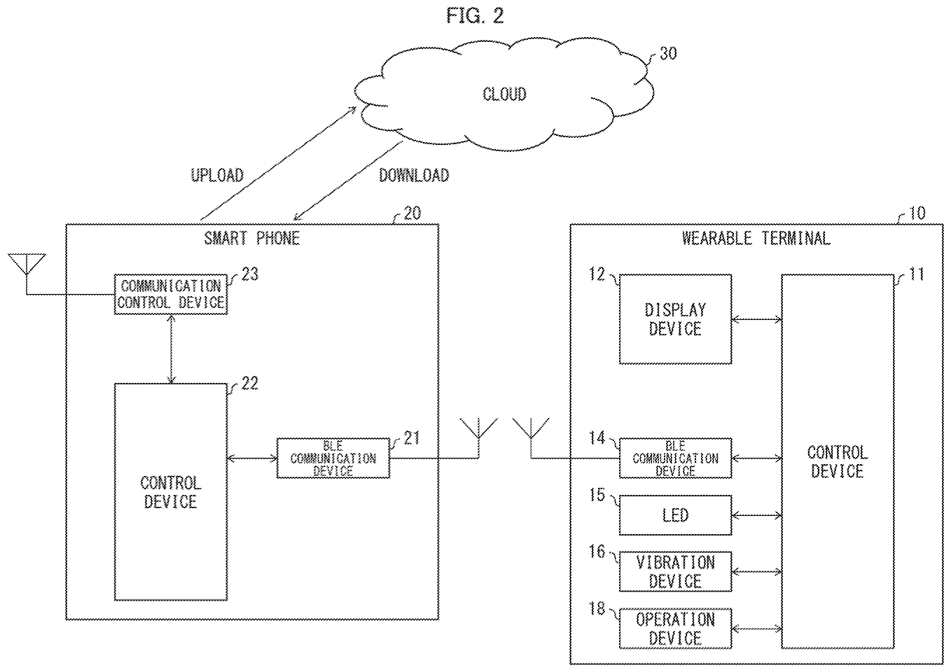

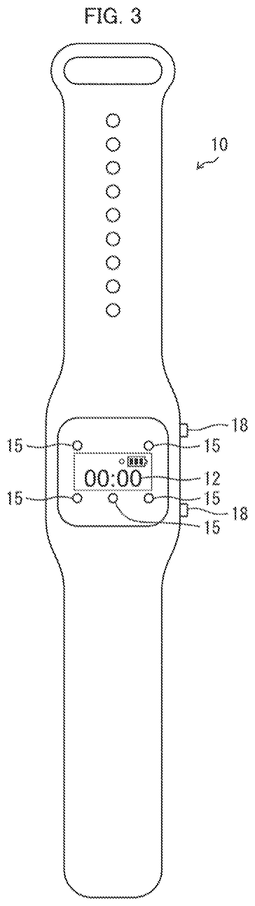

FIG. 2 is a block diagram illustrating respective configurations of a wearable terminal (electronic apparatus) 10 and a smart phone 20 in accordance with Embodiment 1. FIG. 3 is a schematic view illustrating an appearance of the wearable terminal 10 in accordance with Embodiment 1.

(Configuration of Wearable Terminal 10)

The wearable terminal 10 is, for example, a wristwatch-type wearable terminal. The wearable terminal 10 includes at least one control device 11, a display device (information presenting device) 12, a Bluetooth low energy (BLE) communication device 14, an LED (information presenting device, display device) 15, a vibration device (information presenting device) 16, and an operation device 18 (see FIG. 2).

Note that the wearable terminal 10 is not limited to such a wristwatch-type wearable terminal, provided that the wearable terminal 10 is an electronic apparatus. Moreover, the wearable terminal 10 only needs to include at least one information presenting device. That is, the wearable terminal 10 only needs to include, for example, at least one of the display device 12, the LED 15, and the vibration device 16. The wearable terminal 10 can further include a device (information presenting device), which is different from the above three devices and carries out an information presenting process.

The control device 11 is an arithmetic device that has a function to comprehensively control constituent elements of the wearable terminal 10. The control device 11 controls at least one of the display device 12, the LED 15, and the vibration device 16 to present information in response to a situation of a game which situation has been determined based on information that (i) relates to the game on which a user is cheering and (ii) is accumulated in a cloud (server) 30. Note that, specifically, the control device 11 carries out a digitization with respect to the intensity of user's cheering so as to indicate the situation of the game, and the control device 11 controls the information presenting device to present information in accordance with a digitized intensity.

Note that the control device 11 in accordance with Embodiment 1 is not limited to the controlling process of causing the presenting of information in accordance with a situation of a game on which the user is cheering, provided that the presenting of information is caused in accordance with a situation of a game which the user is watching and reacting to.

The control device 11 is specifically realized by a central processing unit (CPU) or the like. The control device 11 will be later described in detail. The display device 12 displays characters, an image, and/or the like, and can be realized, for example, by a liquid crystal display or the like (see FIG. 3).

The BLE communication device 14 is configured to control BLE, which is a radio communication protocol, and establishes a radio communication with a BLE communication device 21 included in the smart phone 20. The LED 15 is controlled, by the control device 11, to change (i) an emission color and (ii) an intensity of light so as to present information. The LED 15 is, for example, provided around the display device 12 as illustrated in FIG. 3. The vibration device 16 is realized, for example, by a vibrator. Specifically, the vibrator is controlled, by the control device 11, to change its pattern and its intensity so as to present information. The operation device 18 supplies information to the control device 11 in response to an operation conducted by the user. Specifically, the operation device 18 can be, for example, a button or the like that is provided on the right hand side of the display device 12 (see FIG. 3).

(Configuration of Smart Phone 20)

The smart phone 20 is a multifunctional mobile phone that functions as a personal computer, a personal digital assistant (PDA), and the like. The smart phone 20 includes the BLE communication device 21, a control device 22, and a communication control device 23 (see FIG. 2). The BLE communication device 21 controls BLE which is a radio communication protocol, and makes a radio communication with the BLE communication device 14 which is included in the wearable terminal 10. The control device 22 is an arithmetic unit that has a function to comprehensively control constituent elements of the smart phone 20. Specifically, the control device 22 is a CPU or the like. The communication control device 23 is a device for sending and receiving (i.e., uploading and downloading) data between the smart phone 20 and the cloud 30.

(Cloud 30)

The cloud 30 stores therein various kinds of data in relation to user's favorite team and players. The cloud 30 is a server that sends/receives various kinds of data to/from the smart phone 20.

(Configuration of Control Device 11)

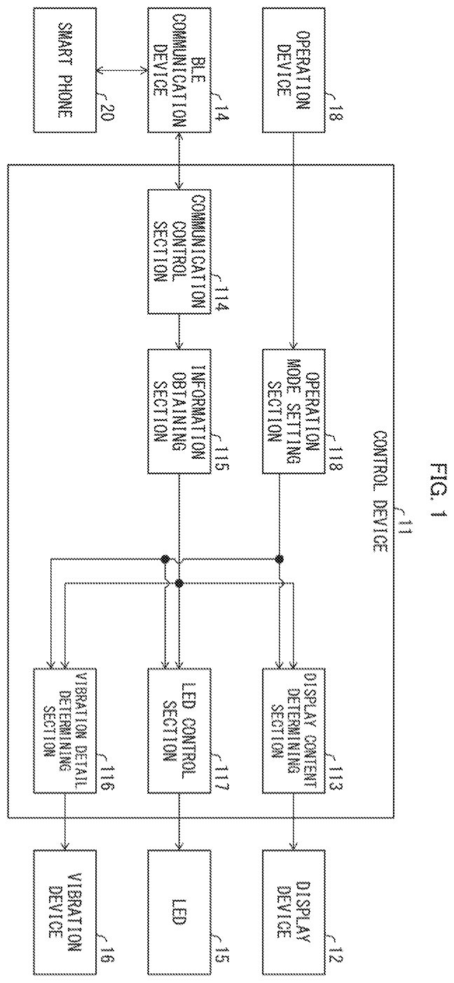

FIG. 1 is a block diagram illustrating a main part configuration of the control device 11, which is included in the wearable terminal 10, in accordance with Embodiment 1. The control device 11 includes a display content determining section 113, a communication control section 114, an information obtaining section 115, a vibration detail determining section 116, an LED control section 117, and an operation mode setting section 118 (see FIG. 1).

The display content determining section 113 determines, based on a situation of a game and an operation mode, display content to be displayed on the display device 12. The situation of the game is determined based on information that (i) relates to the game on which the user is cheering and (ii) has been accumulated in the cloud 30, and the operation mode is set based on information obtained from the operation device 18. Then, the display content determining section 113 causes the display device 12 to display the display content. The communication control section 114 transmits receives data to/from the smart phone 20 via the BLE communication device 14. The information obtaining section 115 obtains the information that (i) relates to the game on which the user is cheering and (ii) is accumulated in the cloud 30, and then supplies the information to the display content determining section 113, the vibration detail determining section 116, and the LED control section 117.

The vibration detail determining section 116 determines vibration details of the vibration device 16 based on a situation of a game and an operation mode. The situation of the game is determined based on information that (i) relates to the game on which the user is cheering and (ii) has been accumulated in the cloud 30, and the operation mode is set based on information obtained from the operation device 18. Then, the vibration detail determining section 116 causes the vibration device 16 to vibrate in accordance with the vibration details. The LED control section 117 determines a light emission manner of the LED 15 based on a situation of a game and an operation mode. The situation of the game is determined based on information that (i) relates to the game on which the user is cheering and (ii) has been accumulated in the cloud 30, and the operation mode is set based on information obtained from the operation device 18. The operation mode setting section 118 sets an operation mode based on information obtained from the operation device 18, and then supplies the operation mode to the display content determining section 113, the vibration detail determining section 116, and the LED control section 117.

(Process by Control Device 11)

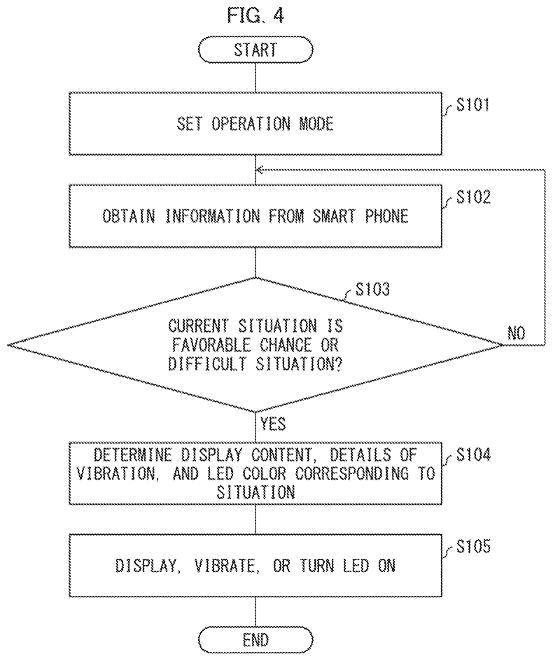

FIG. 4 is a flowchart showing processes carried out by the control device 11 that is included in the wearable terminal 10 in accordance with Embodiment 1.

(Step S101)

In the control device 11 of the wearable terminal 10, first, the operation mode setting section 118 obtains information which has been supplied from the operation device 18 in accordance with an operation conducted by the user. Then, the operation mode setting section 118 sets an operation mode in which the user's favorite team is indicated and then (ii) supplies the operation mode to the display content determining section 113 and the vibration detail determining section 116.

(Step S102)

Next, the information obtaining section 115 obtains information, which (i) relates to the game on which the user is cheering and (ii) is accumulated in the cloud 30, from the smart phone 20 via the BLE communication device and the communication control section 114. The information includes (i) information, related to the current game, such as the number of outs, a score, information about whether a runner is on base and (ii) information in relation to a batter's past such as a won-lost record with respect to a pitcher, a batting average with runners in scoring position, a slugging average, an on-base percentage, and a record for each baseball field, and a record for each day of the week.

(Step S103)

Then, the information obtaining section 115 determines, with the use of the information in relation to the current game, whether or not a current situation gives a favorable chance or tells a difficult situation.

In a case where the information obtaining section 115 has determined that the current situation is a favorable chance or a difficult situation (Yes in step S103), the information obtaining section 115 carries out a digitization, based on, for example, the number of outs and the information about whether a runner is on base, with respect to (i) a degree of the difficult situation (inferiority) or (ii) a degree of the favorable chance (superiority) of the favorite team, each of (i) and (ii) serving as an intensity of cheering. Specifically, the information obtaining section 115 can carry out a digitization with respect to a plurality of degrees of the situation i.e., (i) a very difficult situation, (ii) a difficult situation, (iii) a favorable chance, and (iv) a big chance, each of (i) through (iv) serving as an intensity of cheering. The information obtaining section 115 supplies a digitized degree of the situation to the display content determining section 113, the vibration detail determining section 116, and the LED control section 117.

Note that such a digitization is, of course, not limited to the four levels (i.e., the very difficult situation, the difficult situation, the favorable chance, and the big chance). Alternatively, it is possible to set a larger number of levels. The information, which is used in digitization for indicating inferiority or superiority, is not limited to (i) the number of outs and (ii) the information about whether a runner is on base. Alternatively, it is possible to use a won-lost record between a batter and a pitcher, a batting average with runners in scoring position, and a slugging average.

In a case where the information obtaining section 115 has determined that the current situation is neither a favorable chance nor a difficult situation (Yes in step S103), the information obtaining section 115 returns to the step S102, and obtains again information that (i) relates to the game on which the user is cheering and (ii) is accumulated in the cloud 30.

(Step S104)

Each of the display content determining section 113, the vibration detail determining section 116, and the LED control section 117 obtains, from the operation mode setting section 118, an operation mode in which a user's favorite team is indicated. Further, each of the display content determining section 113, the vibration detail determining section 116, and the LED control section 117 also obtains, from the information obtaining section 115, a digitized degree. Then, the display content determining section 113, the vibration detail determining section 116, and the LED control section 117 determine, in accordance with a situation of the game, display content to be displayed on the display device 12, vibration details of the vibration device 16, and a light emission manner of the LED 15, respectively. The following description will discuss their examples.

For example, the display content determining section 113 determines display content (e.g., “Very difficult situation!!”, “Difficult situation!”, “Favorable chance!” “Big chance!!”, or the like) to be displayed on the display device 12 in accordance with a degree of difficult situation or favorable chance of the user's favorite team.

The vibration detail determining section 116 determines an intensity and intervals of vibration of the vibration device 16 depending on a degree of difficult situation or favorable chance of the user's favorite team.

The LED control section 117 determines a color of the LED 15 which color is appropriate to a team color of the user's favorite team. Further, the LED control section 117 determines an appropriate turn-on pattern, turn-off pattern, or blinking pattern of the LED 15 in accordance with a degree of difficult situation or favorable chance of the user's favorite team. In a case where (i) an event (such as getting of a hit or score) that is favorable to the user's favorite team has occurred or (ii) a favorable chance has come, the LED control section 117 determines a color of the LED 15 which is appropriate to the team color of the user's favorite team. In contrast, in a situation which is advantageous to an opposing team for the user, the LED control section 117 can determine a color of the LED 15 which color is appropriate to a team color of the opposing team.

(Step S105)

Each of the display content determining section 113, the vibration detail determining section 116, and the LED control section 117 controls a corresponding information presenting device to present information in accordance with a digitized degree. That is, the display content determining section 113 controls the display device 12 to display the display content which has been determined. Next, the vibration detail determining section 116 controls the vibration device 16 to vibrate based on the vibration details which have been determined. Then, the LED control section 117 controls the LED 15 to be turned on or off or to blink in the color and the pattern which have been determined (that is, the LED control section 117 controls the LED 15 to light up in a color that varies in accordance with the situation of the game).

### Effect of Embodiment 1

With the configuration of Embodiment 1, first, information in relation to the current game and information in relation to a batter's past are used. This makes it possible to provide representations that accurately correspond to a difficult situation or a favorable chance. Moreover, it is possible to create higher intensity of cheering and an increasing sense of unity for the user, by (i) sharing feelings with respect to a difficult situation or a favorable chance among fans in a baseball field, (ii) a color of the LED 15 that is selected in consideration of the team color of the user's favorite team, and (iii) a representation that is achieved by vibration in line with the difficult situation or the favorable chance. Furthermore, it is possible to cause the user to recognize pleasure of cheering in the baseball field, and this makes it possible to motivate the user to go to the baseball field.

Team colors strongly remind people of their respective baseball teams (e.g., red color reminds people of team A, yellow color reminds people of team B, and the like). Therefore, in a case where the LED 15 is controlled to light up in colors in conformity with the respective team colors, it is possible for the user to create a sense of unity with his/her favorite baseball team.

For example, in a pinch, the LED 15 continues to light up in a team color of the opposing team. This stirs up the user's anxiety. In contrast, in a case where a fine play causes the favorite team to have got out of such a pinch by, the LED 15 lights up in the team color of the favorite team, and this makes it possible to create a sense of relief for the user. Even users and children who are not familiar with baseball can easily recognize whether the favorite team is in an advantageous situation or in a disadvantageous situation, based on the color of the LED 15 which color corresponds to a team color of their favorite team. This allows such users and children to concentrate on cheering with less perplexity.

Embodiments 1 and 2 describe an example in which a cheering motion is carried out to show favor toward a particular subject. Note, however, that Embodiments 1 and 2 are not limited to such an example. For example, the technique in accordance with Embodiments of the present invention can address an unfavorable motion with respect to an unfavorite team or can address an unfavorable motion even with respect to a favorite team. Alternatively, the technique in accordance with Embodiments of the present invention can address entire motions of the user's body, instead of the motion of the user's body in watching a sporting event.

### Embodiment 2

The following description will discuss Embodiment 2 of the present invention. For convenience, identical reference numerals are given to constituent members having functions identical with those of the constituent members described in Embodiment 1, and descriptions of such constituent members are omitted here.

A control device 11 in accordance with Embodiment 2 uploads an intensity of cheering to a cloud 30 via a smart phone 20.

The cloud 30 obtains the intensity of cheering from the control device 11 via the smart phone 20, and carries out a digitization with respect to the intensity of cheering thus obtained (e.g., assigns the intensity of cheering to any of four levels, i.e., a very difficult situation, (ii) a difficult situation, (iii) a favorable chance, and (iv) a big chance). Then, the cloud 30 transmits the numerical value, which indicates the intensity of cheering, to the control device 11 via the smart phone 20.

The control device 11 receives the numerical value, which indicates the intensity of cheering, from the cloud 30 via the smart phone 20, and then controls each of a display device 12, an LED 15, and a vibration device 16 to reflect the numerical value in presenting of information.

Moreover, the cloud 30 simultaneously transmits the numerical value, which indicates the intensity of cheering, to smart phones 20 of the users who are cheering on one team. Each of the smart phones 20 receives the numerical value received from the cloud 30, and transmits the numerical value to a corresponding one of wearable terminals 10.

Then, the control device 11 controls the display device 12, the LED 15, and/or the vibration device 16 to present information, in accordance with the numerical value (i.e., information) that has been simultaneously transmitted from the cloud 30, via the respective smart phones 20, to the wearable terminals 10 of the users who are cheering on the one team.

Note that the control device 11, the smart phone 20, and the cloud 30 in accordance with Embodiment 2 are not limited to a process that relates to the intensity of cheering of the user, provided that the process relates to an intensity of reaction of the user.

### Effect of Embodiment 2

Embodiment 2, a numerical value that indicates an intensity of cheering is reflected by the wearable terminal 10. With the configuration, an effect of increasing a sense of anticipation with respect to a favorable chance is brought about, and an effect of increasing a sense of crisis in a difficult situation is brought about. As such, it is possible to expect a further synergistic effect.

In a case where a numerical value that indicates an intensity of cheering on one team is simultaneously transmitted to smart phones 20 and further to wearable terminals 10 of users who are cheering on the one team, it is possible to create a sense of unity among fans in a baseball field, and this leads to a greater intensity of cheering.

[Example of Configuration Achieved by Software]

The control blocks particular, the display content determining section 113, the vibration detail determining section 116, and the LED control section 117) of the control device 11 can be realized by a logic circuit (hardware) provided in an integrated circuit (IC chip) or the like or can be alternatively realized by software.

In the latter case, the control device 11 includes a computer that executes instructions of a program that is software realizing the foregoing functions. The computer includes, for example, at least one processor and a computer-readable storage medium storing the program. In a case where the processor in the computer reads out the program from the storage medium and executes the program, the object of the present invention is achieved. The processor can be, for example, a CPU. The storage medium can be a “non-transitory tangible medium” such as, for example, a tape, a disk, a card, a semiconductor memory, a programmable logic circuit, or the like, as well as a read only memory (ROM) or the like. The computer can further include a random access memory (RAM) in which the program is loaded. The program can be made available to the computer via any transmission medium (such as a communication network or a broadcast wave) that can transmit the program. Note that an aspect of the present invention can also be achieved in the form of a computer data signal in which the program is embodied via electronic transmission and which is embedded in a carrier wave.

Aspects of the present invention can also be expressed as follows:

The electronic apparatus (wearable terminal 10) in accordance with an aspect 1 of the present invention includes an information presenting device (display device 12, LED 15, vibration device 16); and a control device (11), the control device controlling the information presenting device to present information in response to a situation of a game which situation has been determined based on information that (i) relates to the game which a user is watching and reacting to and (ii) is accumulated in a server (cloud 30).

According to the configuration, the control device uses the information that (i) relates to the game which the user is watching and reacting to and (ii) is accumulated in the server. This makes it possible to present appropriate information that corresponds to a situation of the game.

According to the electronic apparatus in accordance with an aspect 2 of the present invention, it is possible in the aspect 1 that the control device carries out a digitization with respect to an intensity of reaction of the user so as to indicate the situation of the game, and the control device controls the information presenting device to present information in accordance with a digitized intensity of reaction.

According to the configuration, the control device carries out a digitization with respect to the intensity of reaction of the user, and this makes it possible to present more appropriate information that corresponds to the situation of the game.

According to the electronic apparatus in accordance with an aspect 3 of the present invention, it is possible in the aspect 2 that the control device carries out a digitization with respect to a degree of inferiority or superiority of a team, which the user is supporting, so as to indicate the intensity of reaction, and the control device controls the information presenting device to present information in accordance with a digitized degree.

According to the configuration, the control device carries out digitization with respect to the degree of inferiority or superiority of the team which the user is supporting, and this makes it possible to present more appropriate information that corresponds to the intensity of reaction.

According to the electronic apparatus in accordance with an aspect 4 of the present invention, it is possible in any of the aspects 1 through 3 that the information presenting device is a display device (display device 12, LED 15), and the control device controls the display device to display a color that varies in accordance with the situation of the game.

According to the configuration, the control device controls the display device to display a color that varies in accordance with the situation of the game which the user is watching and reacting to, and this makes it possible to present clear information that corresponds to the situation of the game.

According to the electronic apparatus in accordance with an aspect 5 of the present invention, it is possible in any of the aspects 1 through 4 that the control device controls the information presenting device to present the information based on information which has been simultaneously transmitted from the server to electronic apparatuses of users who support one team.

According to the configuration, the control device uses the information which has been simultaneously supplied from the server to the electronic apparatuses of users who support one team, and this makes it possible to create a sense of unity among the users in accordance with the situation of the game.

The control device in accordance with an aspect 6 of the present invention is a control device for controlling an electronic apparatus and controls an information presenting device included in the electronic apparatus to present information in response to a situation of a game which situation has been determined based on information that (i) relates to the game which a user is watching and reacting to and (ii) is accumulated in a server.

According to the configuration, the information that (i) relates to the game which the user is watching and reacting to and (ii) is accumulated in the server is used with respect to the electronic apparatus, and it is therefore possible to provide the control device that causes appropriate information corresponding to the situation of the game to be presented.

A control method in accordance with an aspect 7 of the present invention is a method for controlling an electronic apparatus and includes the step of controlling an information presenting device included in the electronic apparatus to present information in response to a situation of a game which situation has been determined based on information that (i) relates to the game which a user is watching and reacting to and (ii) is accumulated in a server.

According to the configuration, the information that relates to the game which the user is watching and reacting to and is accumulated in the server is used with respect to the electronic apparatus, and it is therefore possible to provide the control method that causes appropriate information corresponding to the situation of the game to be presented.

A control device in accordance with each aspect of the present invention can be realized by a computer. The computer is operated based on (i) a program for causing the computer to realize the control device by causing the computer to operate as each section (software element) included in the control device and (ii) a computer-readable storage medium in which the program is stored. Such a program and a computer-readable storage medium are included in the scope of the present invention.

[Additional Remarks]

The present invention is not limited to the embodiments, but can be altered by a skilled person in the art within the scope of the claims. The present invention also encompasses, in its technical scope, any embodiment derived by combining technical means disclosed in differing embodiments. Further, it is possible to form a new technical feature by combining the technical means disclosed in the respective embodiments.

### REFERENCE SIGNS LIST

10: Wearable terminal (electronic apparatus)

11: Control device

12: Display device (information presenting device)

15: LED (information presenting device, display device)

16: Vibration device (information presenting device)

30: Cloud (server)

1. An electronic apparatus, comprising:
an information presenting device; and
a control device,
the control device controlling the information presenting device to present information in response to a situation of a game which situation has been determined based on information that (i) relates to the game which a user is watching and reacting to and (ii) is accumulated in a server.

2. The electronic apparatus as set forth in claim 1, wherein the control device carries out a digitization with respect to an intensity of reaction of the user so as to indicate the situation of the game, and the control device controls the information presenting device to present information in accordance with a digitized intensity of reaction.

3. The electronic apparatus as set forth in claim 2, wherein the control device carries out a digitization with respect to a degree of inferiority or superiority of a team, which the user is supporting, so as to indicate the intensity of reaction, and the control device controls the information presenting device to present information in accordance with a digitized degree.

4. The electronic apparatus as set forth in claim 1, wherein:
the information presenting device is a display device; and
the control device controls the display device to display a color that varies in accordance with the situation of the game.

5. The electronic apparatus as set forth in claim 1, wherein the control device controls the information presenting device to present the information based on information which has been simultaneously transmitted from the server to electronic apparatuses of users who support one team.

6. A computer-readable non-transitory recording medium on which a program for causing a computer to serve as a control device included in an electronic apparatus recited in claim 1 is stored.

7. A control device for controlling an electronic apparatus, wherein said control device controls an information presenting device included in the electronic apparatus to present information in response to a situation of a game which situation has been determined based on information that (i) relates to the game which a user is watching and reacting to and (ii) is accumulated in a server.

8. A method for controlling an electronic apparatus, said method comprising the step of:
controlling an information presenting device included in the electronic apparatus to present information in response to a situation of a game which situation has been determined based on information that (i) relates to the game which a user is watching and reacting to and (ii) is accumulated in a server.
